(19) United States
(12) Patent Application Publication
(10) Pub. No.: US 2017/0296224 A1
(43) Pub. Date: Oct. 19, 2017

(54) DEVICE FOR A MEDICAL PROCEDURE, COMPRISING A TROCAR AT ONE END AND A CANNULA AT THE OTHER END

(71) Applicant: NEEDLE CONCEPT, BIARRITZ (FR)

(72) Inventors: Olivier BUCHS, PEYREHORADE (FR); Grégory DJIANE, SAINT GRATIEN (FR)

(73) Assignee: NEEDLE CONCEPT, BIARRITZ (FR)

(21) Appl. No.: 15/638,939

(22) Filed: Jun. 30, 2017

Related U.S. Application Data
(63) Continuation of application No. PCT/FR2016/050019, filed on Jan. 6, 2016.

(30) Foreign Application Priority Data
Jan. 6, 2015 (FR) 15/50072

(51) Int. Cl. A61B 17/34; A61M 5/00; A61B 17/3209

(52) U.S. Cl. CPC A61B 17/34; A61M 5/002; A61B 17/32093

(57) ABSTRACT

A device for a medical procedure is provided that includes a handle axially supporting on one side a rigid needle forming a trocar provided to pierce skin, a sealing plug covering at least the rigid needle, and an elongated protective tube mounted on the handle on another side of said handle, said elongated protective tube containing a needle forming a cannula.

### CROSS-REFERENCE TO RELATED APPLICATIONS

This application is a continuation of International Application No. PCT/FR2016/050019, filed on Jan. 6, 2016, which claims priority to and the benefit of FR 15/50072 filed on Jan. 6, 2015. The disclosures of the above applications are incorporated herein by reference.

### FIELD

The present disclosure relates to a device supporting both a rigid needle forming a trocar as well as a generally flexible needle forming a cannula provided for carrying out surgical operations.

### BACKGROUND

The statements in this section merely provide background information related to the present disclosure and may not constitute prior art.

In order to carry out some surgical operations such as injections, punctures or biopsies, it is known to use an instrument called a trocar, including a handle extended by a sharp stem allowing piercing the skin of the patients.

Then, a cannula is installed in the piercing, which cannula is formed by a generally relatively flexible tube comprising, at its front end, an orifice inserted into the body at the desired location and, at its rear end, a mouthpiece allowing linking it in particular to a syringe or to a pouch for injections or infusions, or to a means performing samplings.

In this case, the operator uses two distinct tools to perform these two operations successively. It is then necessary to independently prepare and sterilize these two tools, then to pack up and package them in order to send them to care centers. These separate operations imply relatively significant costs, and give by these individual packages a high overall dimension which occupies space and increases the fees.

In addition, in the care center, it is then necessary to supply to the workstation the two tools that the operator must successively unpack from its sterile packaging in order to use them, one at a time. Then, after the first piercing operation with the trocar, the operator must put it down, then grasp the cannula to insert it into the body, which makes successive manipulations.

In particular, after putting down the trocar with its needle in the open air, there is a risk of stick-incident with this needle during the following manipulations by the operator or by the accompanying personnel, in particular during the removal of the tools after the operation.

### SUMMARY

The present disclosure provides a device for a medical procedure, comprising a handle axially supporting, on one side, a rigid needle forming a trocar provided to pierce the skin, the device being equipped with a sealing plug covering at least the rigid needle, remarkable in that it includes, on the other side, an elongated protective tube mounted on this handle, containing a needle forming a cannula, that is to say remarkable in that the device includes an elongated protective tube mounted on the handle on the other side of said handle, said elongated protective tube containing a needle forming a cannula.

An advantage of this device is that, by providing, on each side in a protected manner, for the rigid needle forming a trocar as well as the flexible needle forming a cannula, a sub-assembly is made, comprising the tools for the two successive operations, having a single sterilization operation and receiving a compact packaging, thereby facilitating the manipulations and reducing costs.

In addition, the preparation of the workstation is facilitated by the supply of a single device which allows, after the first piercing operation of the skin, to quickly put back in place the plug protecting the rigid needle, thereby protecting it, and then to remove the cannula from its protective tube by turning the handle, in order to use it for the next injection or sampling operation.

The device for medical procedure, for example a surgical operation, according to the present disclosure may additionally include one or more of the following characteristics, which can be combined together.

Advantageously, the device comprises a protective cap mounted or fastened on the handle, the protective cap being covered by the plug and covering the rigid needle.

The handle may include a substantially cylindrical front portion containing an axial piercing receiving the rear end of the rigid needle, which receives and maintains the cap by clamping.

Advantageously, the cap includes, at the front end, an outer circular bead having a flat front face. The bead facilitates a traction on the cap for extracting it, and in addition widens the flat face giving a better stability to this cap placed in an upstanding position.

Advantageously, the plug covers the cap and the handle, and has a substantially cylindrical portion clamping the protective tube. Thus, a maintenance of the sterilization of the whole of the inner device is carried out by means of a single clamping which must be sealed to preserve the sterility inside.

In this case, advantageously the clamping surfaces of the protective tube and of the plug on one another are formed by a plastic molding in a continuous circular mold portion giving absence of burrs. This absence of burrs avoids passages which may allow germs of infection to enter.

Advantageously, the protective tube and the plug include outer axial ribs or ridges. These ribs and these ridges facilitate the handling of the components, as well as a rotation and a traction allowing opening the plug.

Particularly, the protective tube may include four ribs disposed in a crossing manner, which extend substantially over the entire length of the tube containing the cannula. These ribs provide, with a reduced amount of material, a significant reinforcement of the tube.

Advantageously, the device includes axial ribs providing, on their outer contours, the clamping of a cannula holder in the protective tube, of the protective tube in the handle, or of the handle in the cap. The clamping of the two elements, facilitating the disassembling, is thus limited.

Advantageously, the device includes a cannula holder having to the front an outer thread comprising at least two different superposed pitches. It is thus possible to easily fasten this cannula holder on different models of syringes usually marketed.

Advantageously, the different molded components of this device are formed by a molding of a plastic material comprising shapes allowing an axial demolding.

Further areas of applicability will become apparent from the description provided herein. It should be understood that the description and specific examples are intended for purposes of illustration only and are not intended to limit the scope of the present disclosure.

### DRAWINGS

In order that the disclosure may be well understood, there will now be described various forms thereof, given by way of example, reference being made to the accompanying drawings, in which:

FIG. 1 a side view of a device for surgical operation according to the teachings of the present disclosure;

FIG. 2 is a side cross-sectional view of the device of FIG. 1;

FIG. 3 is a perspective view of the device of FIG. 1;

FIG. 4 is the device of FIG. 1 at the start of a sequence of operations having a removed plug in accordance with the teachings of the present disclosure;

FIG. 5 is the device of FIG. 1 at the next sequence of operations having a removed cap of the rigid needle in accordance with the teachings of the present disclosure;

FIG. 6 is the device of FIG. 1 at the next sequence of operations having a replaced cap in accordance with the teachings of the present disclosure;

FIG. 7 is the device of FIG. 1 at the next sequence of operations illustrating the handle of the removed trocar in accordance with the teachings of the present disclosure;

FIG. 8 is the device of FIG. 1 at the next sequence of operations illustrating the protective tube of the cannula removed with a placed syringe in accordance with the teachings of the present disclosure;

FIG. 9 is a side view of a device according to another form of the present disclosure;

FIG. 10 is a perspective view of the device of FIG. 9 having a removed plug in accordance with the teachings of the present disclosure; and

FIG. 11 is a perspective view of the device of FIG. 9 having a removed cap of the rigid needle in accordance with the teachings of the present disclosure.

The drawings described herein are for illustration purposes only and are not intended to limit the scope of the present disclosure in any way.

### DETAILED DESCRIPTION

The following description is merely exemplary in nature and is not intended to limit the present disclosure, application, or uses. It should be understood that throughout the drawings, corresponding reference numerals indicate like or corresponding parts and features.

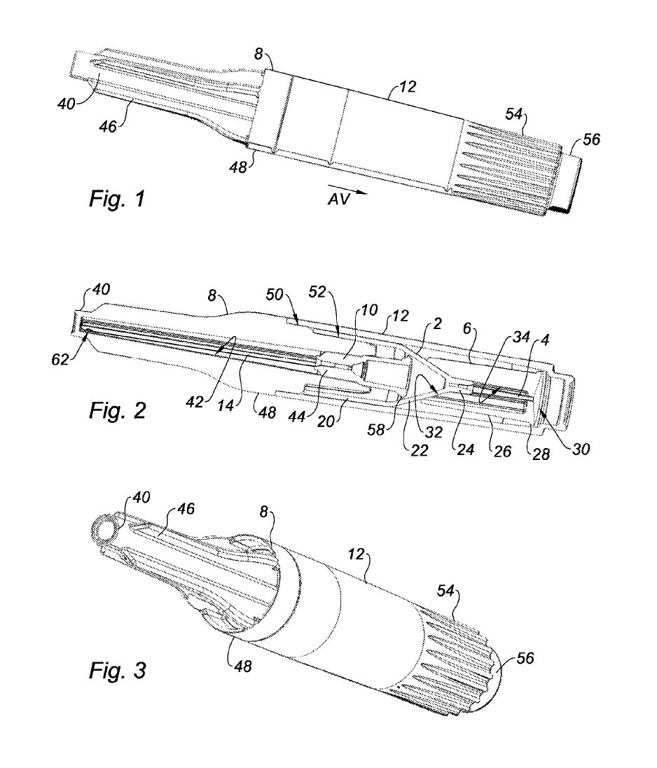

FIGS. 1, 2 and 3 illustrate a device for a medical procedure such as a surgical operation forming an assembly elongated along a main axis, comprising an inner handle 2 supporting on the front side indicated by the arrow noted “AV,” a rigid needle 4 forming a trocar provided to pierce the skin of a patient. It is meant by the term “trocar” an assembly comprising a handle to which is fastened a rigid and piercing needle or stem.

The rigid needle is protected by a cap 6 fastened around the handle 2, this assembly is itself protected by a sealing plug 12 also fastened here around a protective tube 8.

The handle 2 receives, on the rear side, the protective tube 8 which is fastened inside. The protective tube 8 receives on the front side a cannula holder 10 clamped in this tube, which supports a cannula 14 extending up to the rear end of this tube.

In one form, all the components of the device are made by a molding by injection of a plastic material, advantageously including polypropylene or polycarbonate. The rigid needle 4 as well as the cannula 14 are made of stainless steel.

The handle 2 includes a substantially cylindrical hollow circular portion 20 extending forwardly by a tapered conical portion 22 which ends in a substantially cylindrical shape 24 receiving, in a front axial piercing, the rear end of the rigid needle 4.

The rigid needle 4 includes a rear end provided to be clamped in the front axial piercing of the handle 2, and a front tip which can include different shapes commonly used for the trocars, comprising in particular a cross transverse section ending with a tip facilitating the piercing of the skin.

The cap 6 includes a substantially cylindrical outer shape 26, ending at the front end by an outer circular bead 28, having a flat front face 30 comprising a small inner hollow member. This flat front face 30 allows, after having removed the plug 12, to place the device in an upstanding position, the outer bead 28 ensuring a larger base giving it more stability in this position as well as a better adhesion for the cap 6 in the assembled position.

The cap 6 includes inside, starting from the rear side, a tapered conical hollow member 32, then a substantially cylindrical piercing 34 which extends almost up to the end, receiving the rigid needle 4. The piercing 34 includes, on its inner wall, axial ribs extending over the entire length, which reduce the amount of material.

The rear portion of the cylindrical hollow member 34 fits on the front cylindrical shape 24 of the handle 2, to be clamped thereon and to maintain the cap 6 in place during its assembling, the axial ribs of this hollow member limiting the clamping. The outer bead of the cap 28 constitutes a shape allowing a traction thereon to facilitate its removal from the handle 2.

The substantially cylindrical shapes of the cap 6 include a small slope providing to the rear a tightening of the diameter of the outer cylindrical shape 26 and an increase in the diameter of the inner piercing 34, constituting relief angles allowing an axial demolding of the cap from a mold comprising circular shapes in a single portion which are removed by sliding rearwards.

The protective tube 8 includes, starting from the rear, a substantially cylindrical outer shape 40 of small diameter, containing inside a substantially cylindrical piercing 42 opening forwardly, receiving the cannula 14. The cylindrical piercing 42 includes on its inner wall, small axial ribs extending over its entire length which reduce the amount of material.

The cannula holder 10 ends to the rear with axial ribs 44 having edges forming a substantially cylindrical outer contour, receiving the clamping of the piercing 42 of the protective tube 8, which is smooth in this portion. The support on the edges of the ribs 44 allows to limit the clamping, and facilitates the loosening.

The cannula holder 10 extends forwardly by a substantially cylindrical portion ending with an outer thread 58 comprising two different superimposed pitches of 1.25 and 2.5 mm allowing the screwing of a commercial syringe, which usually presents one of these two pitches.

A front axial piercing of the cannula holder 10 receives the cannula 14 formed by a hollow needle receiving the liquid delivered by the syringe, having a rounded front end including an injection hole on the side 62 so as not to prick the skin or the flesh during its introduction in the piercing previously carried out by the rigid needle 4.

The cylindrical outer shape 40 of the protective tube 8 is reinforced after the rear end by four ribs 46 disposed in a crossing manner in a transverse section, comprising at first a substantially constant height which, to the middle of this tube, has a high-magnification along a regular curve. The outer edges of the ribs 46 are tangent onto the outer surface of a cylindrical hollow ring 48, these ribs extending inside this ring to support it.

The four cross ribs 46 allow to reinforce effectively, with a mass of reduced material, the cylindrical shape 40 constituting the protective tube of the cannula 14 inserted therein. These ribs 46 also allow to facilitate the gripping of the protective tube 8 with the cannula 14 for the cannula holder 10 screwing with the syringe by means of the outer thread 58.

This screwing is also allowed by the axial ribs disposed on the inner wall of the cylindrical piercing 42 of the protective tube 8 which are engaged, in the assembled position, with the axial ribs 44 at the rear of the cannula holder 10. These axial ribs are meshed together in an assembled position, allowing the transmission of forces, and in particular the rotation of the cannula holder 10, and therefore of the outer thread 58, by the rotation of the protective tube 8, which is facilitated by the ribs 46.

Moreover, these axial ribs are arranged in engagement so that, in the case of too large torque, the transmission of forces is no longer assured. This is here allowed because of the employed plastic materials and of the elastic deformations of said ribs. This allows in particular to control the clamping torque between the cannula holder 10 and the syringe. Indeed, once the predetermined clamping torque is exceeded, the rotation of the protective tube no longer causes that of the cannula holder, thus limiting the clamping torque between the cannula holder 10 and the syringe.

The protective tube 8 then includes to the front, after the hollow ring 48, a first diameter narrowing forming a male cylindrical portion 50 receiving the clamping of the plug 12, then a second narrowing comprising front axial ribs 52 having edges forming a substantially cylindrical outer contour receiving the clamping of the handle 2. The support on the edges of the front ribs 52 allows to limit the clamping and facilitates the loosening.

The shapes of the protective tube 8 include for the cylindrical outer shape 40 and the portion of the edges of the ribs 46 of substantially constant height, a small slope providing a tightening rearwardly forming a relief angle enabling an axial demolding/removal of the mold in this direction.

Moreover, the shapes of the protective tube 8 include, for the male cylindrical portion 50 and the outer edge of the front axial ribs 52, a small slope providing a tightening forwardly as well as, for the inner piercing 42, a small slope having an increase forwardly, allowing an axial demolding/removal of the mold in this direction.

The sealing plug 12 includes a substantially cylindrical overall shape of constant thickness comprising, rearwardly, an inner diameter fitting on the male cylindrical portion 50 of the protective tube 8, in order to obtain an effective maintenance as well as a sealing maintaining the sterility inside.

The plug 12 includes, rearwardly on the outside, a series of axial ridges 54, facilitating the handling as well as the adhesion to twist and pull on this plug in order to remove it from the protective tube 8, the other hand clamping this tube by the ribs 46 also facilitating the grip. The plug 12 then includes a cylindrical short portion of reduced diameter 56, forming the end.

The plug 12 includes, for its inner and outer shapes, a small slope having a narrowing forward portion, allowing an axial demolding/removal of the outer mold forwardly and of the inner mold rearwardly.

Advantageously, the outer shape of the protective tube 8 and the inner shape of the plug 12 are made by a mold comprising, at the clamping surfaces of these two components, in the transverse section, a continuous circular shape, which inhibits the production of small axial burrs during molding which is difficult to avoid between the two portions of a mold having a joint plane along an axial plane. Thus, a good clamping as well as a continuous contact are obtained over the entire periphery of these two components, which provides the sealing as well as the maintenance of the inner sterilization.

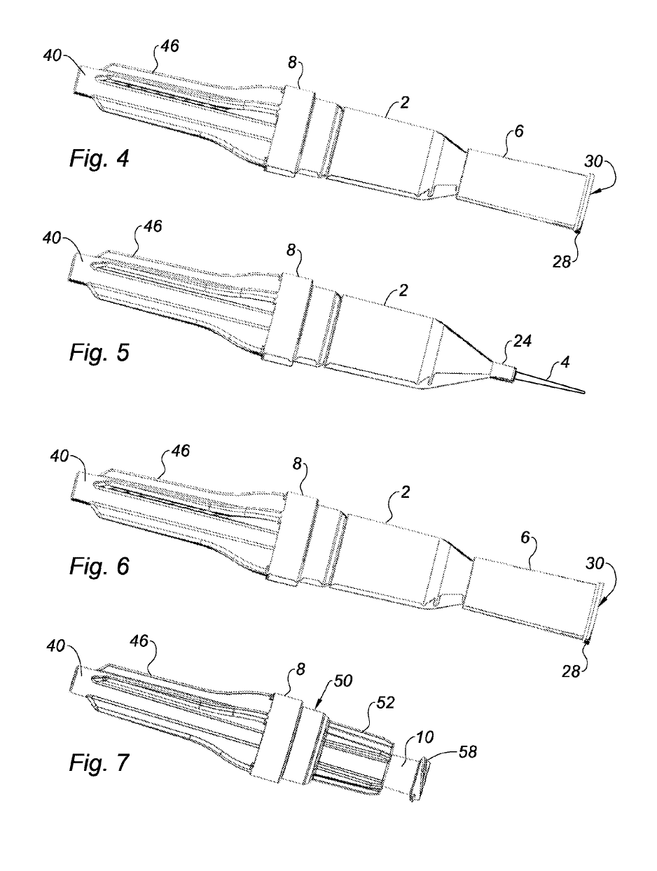

FIG. 4 has a first step of using the device for a medical procedure which has been taken out of its packaging, the operator having removed, at the same place of the operation, the sealing plug 12 by twisting and pulling on its portion comprising the axial ridges 54, to prepare this operation.

It should be noted that the device can be disposed in an upstanding position by placing it on the flat front face 30 of the cap 6 which, being large, ensures good stability.

FIG. 5 has a next step. The operator having removed the cap 6 by pulling on its front outer bead 28, uses the device as a pencil having a good handling thanks to the ribs 46 of the protective tube 8, in order to pierce the skin with the rigid needle 4.

FIG. 6 has a next step, where the operator has replaced the cap 6 on the rigid needle 4 to provide protection of this needle inhibiting the operator from being pricked with it during the following operations comprising the elimination of the tools. The flat front face 30 of the cap 6 then allows to place the cap in an upstanding position.

FIG. 7 has a next step, where the operator has removed the handle 2 from the protective tube 8, which disengages the cannula holder 10 comprising its cannula 14 fastened at its rear end.

FIG. 8 has a next step, where the operator has fastened a syringe 60 on the front end of the cannula holder 10, then removed the protective tube 8 from this cannula holder, which disengages the cannula 14 which becomes operational.

It should be noted that during all operations, the operator keeps handy the two tools comprising the trocar formed by the fixed needle 4 and the cannula 14, which remain protected when they are not in use.

Advantageously, during the manufacture of the device according to the present disclosure, after having assembled all its components, a sterilization of the complete device is carried out by the ethylene oxide which has the advantage of passing through the plastic materials. The prior assembly of this device then does not hinder this sterilization operation. Other sterilization methods may also be used, such as gamma sterilization with suitable doses of rays. In this case, also the sterilization can be performed after having assembled all the components of the device.

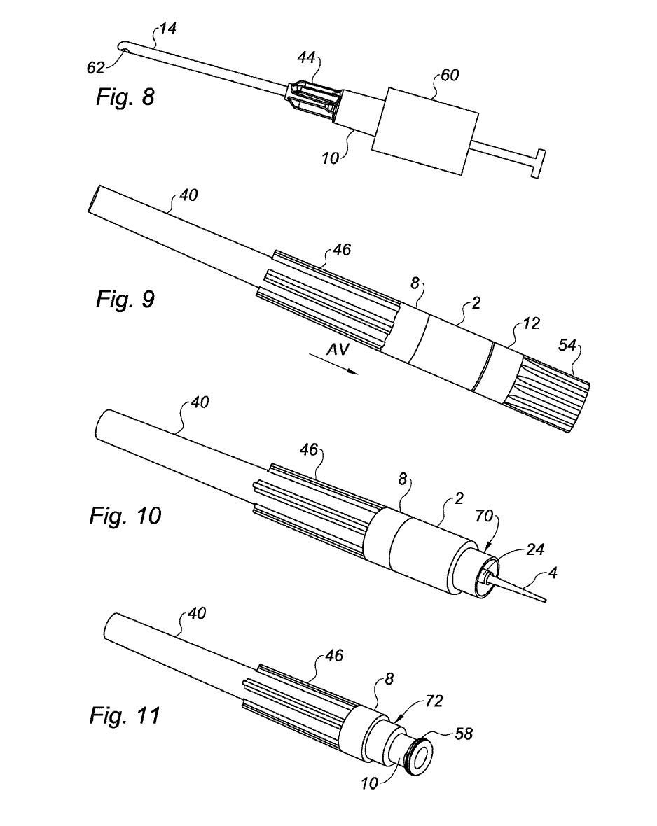

FIGS. 9 to 11 has a device according to a variant comprising substantially the same components, which operates in a similar manner.

In this form, the device includes a sealing plug 12 but does not include a cap 6. The plug 12 also substantially ensures the functions of the cap and ends at the front end by a flat front face allowing to place the plug in an upstanding position with the rigid needle.

The plug 12 comprising the outer axial ridges 54 is short and is mounted or fastened directly on a front male cylindrical portion 70 of the handle 2. The protective tube 8 includes a male cylindrical portion turned towards the front 72, receiving the clamping of the handle 2. It should be noted that in this version, the device includes two clamps instead of one for the preceding version, which is sealed to preserve the sterility inside.

The protective tube 8 includes, starting from the rear, a cylindrical outer portion 40 which is long, the outer ribs 46 beginning only substantially in the middle of this tube.

The cannula holder 10 has at its front end the two outer threads 58 serving for the fastening on the syringe.

The description of the disclosure is merely exemplary in nature and, thus, variations that do not depart from the substance of the disclosure are intended to be within the scope of the disclosure. Such variations are not to be regarded as a departure from the spirit and scope of the disclosure.

What is claimed is:

1. A device for a medical procedure comprising:
a handle axially supporting on one side a rigid needle forming a trocar provided to pierce skin;
a sealing plug covering at least the rigid needle; and
an elongated protective tube mounted on the handle on another side of said handle, said elongated protective tube containing a needle forming a cannula.

2. The device according to claim 1 further comprising a protective cap mounted on the handle, the protective cap being covered by the sealing plug and covering the rigid needle.

3. The device according to claim 2, wherein the handle includes a cylindrical front portion containing an axial piercing receiving a rear end of the rigid needle, the cylindrical front portion configured to receive and maintain the protective cap by clamping.

4. The device according to claim 2, wherein the protective cap includes, at a front end thereof, an outer circular bead having a flat front face.

5. The device according to claim 2, wherein the sealing plug covers the protective cap and the handle and has a cylindrical portion clamping the elongated protective tube.

6. The device according to claim 5, wherein the elongated protective tube and the sealing plug each define clamping surfaces and are formed by a plastic molding in a continuous circular mold portion.

7. The device according to claim 2 further comprising axial ribs having outer contours configured for clamping a cannula holder in at least one of the elongated protective tube, the elongated protective tube in the handle, and the handle in the protective cap.

8. The device according to claim 1, wherein the elongated protective tube and the sealing plug include outer axial ribs.

9. The device according to claim 8, wherein the elongated protective tube further includes four ribs disposed in a crossing manner, which extend substantially over an entire length of the elongated protective tube containing the cannula.

10. The device according to further comprising a cannula holder having a forward portion with an outer thread comprising two different superimposed pitches.

11. The device according to claim 1, wherein each of the handle, the sealing plug, and the elongated protective tube are formed by molding a plastic material and define shapes configured to allow for axial demolding/removal.
